(19) United States
(12) Patent Application Publication
(10) Pub. No.: US 2021/0123039 A1
(43) Pub. Date: Apr. 29, 2021

(54) NANOSTRUCTURED MAGENTIC SCAFFOLD FOR CONTROLLING STEM CELL DIFFERENTIATION

(71) Applicant: KING ABDULLAH UNIVERSITY OF SCIENCE AND TECHNOLOGY, Thuwal (SA)

(72) Inventors: Jürgen KOSEL, Thuwal (SA); Jose Efrain PEREZ, Thuwal (SA); Jasmeen Sayed MERZABAN, Thuwal (SA)

(21) Appl. No.: 17/041,761

(22) PCT Filed: Apr. 2, 2019
(86) PCT No.: PCT/IB2019/052699

§ 371 Date: Sep. 25, 2020

Related U.S. Application Data
(60) Provisional application No. 62/654,049, filed on Apr. 6, 2018.

(51) Int. Cl. C12N 13/00; C12N 5/077; C12M 1/12; C12M 1/42

(52) U.S. Cl. CPC C12N 13/00; C12N 5/0654; C12N 2506/1346; C12M 35/02; C12N 2529/00; C12M 25/14

(57) ABSTRACT

Differentiation of a stem cell involves arranging the stem cell on a scaffold having an ordered array of magnetic one-dimensional nanostructures and culturing the stem cell while applying a magnetic field having a frequency to the ordered array of magnetic one-dimensional nanostructures to differentiate the stem cell.

### CROSS-REFERENCE TO RELATED APPLICATIONS

This application claims priority to U.S. Provisional Patent Application No. 62/654,049, filed on Apr. 6, 2018, entitled “MAGNETIC NANOWIRE SUBSTRATES FOR CELL CULTURE,” the disclosure of which is incorporated herein by reference in its entirety.

### BACKGROUND

### Technical Field

Embodiments of the subject matter disclosed herein generally relate to controlling stem cell differentiation using a nanostructured magnetic scaffold that is actuated by a magnetic field having a frequency.

### Discussion of the Background

The use of stem cells is becoming increasingly attractive in cell-based tissue engineering and regeneration research due to the ability of mesenchymal stem cells to differentiate into a number of cell types, including osteoblasts (bone cells), chondrocytes (cartilage cells), myocytes (muscle cells), and adipocytes (fat cells giving rise to marrow adipose tissue). For example, osteoblasts differentiated from mesenchymal stem cells generate mineralized tissue, resembling bone capable of rehabilitating and improving bone regeneration. The transplantation of mesenchymal stem cells has shown great potential in the treatment of osteoporosis and osteogenesis imperfect.

Current experimental bone tissue engineering protocols utilize a combination of biochemical factors that promote the expression of osteogenic markers such as Runx2, osteopontin (OPN), osteocalcin (OCN) and type 1 collagen. However, long treatment times, side effects, variability and cost factors limit their usability. New strategies aim to overcome these issues by exploiting the important role of the extracellular stimuli of the microenvironment on cell fate, with matrix stiffness, its nano- and microscale geometry, and its influence on cell shape being major factors contributing to stem cell fate. Thus, the use of biomaterials in cell-matrix composites as delivery vehicles of mesenchymal stem cells has become the subject of intensive research as an alternative strategy for bone regeneration.

Document [1] discloses the use of dense, vertically aligned iron nanowires (NWs) as a culturing platform of mesenchymal stem cells, showing its feasibility as a differentiation scaffold due to the high biocompatibility of iron nanowires and the cytoskeleton changes it induced on the cells, mainly in the form of altered actin expression, as well as of the actin-anchoring protein vinculin. This culturing platform can achieve expression of osteopontin in about two weeks. Other culturing techniques similarly achieve expression of osteopontin in about two weeks. Longer the culturing times increase costs, result in lower throughput, and higher chances of failure to due contamination, etc.

Thus, there is a need for methods and systems for that can differentiate stems cells in a shorter period of time.

### SUMMARY

According to an embodiment, there is a method for differentiation of a stem cell. The stem cell is arranged on a scaffold having an ordered array of magnetic one-dimensional nanostructures. The stem cell is cultured while applying a magnetic field having a frequency to the ordered array of magnetic one-dimensional nanostructures to differentiate the stem cell.

According to another embodiment there is a system for differentiation of a stem cell. The system includes a scaffold comprising an ordered array of magnetic one-dimensional nanostructures. A magnet is proximate to the scaffold so that a magnetic field produced by the magnet projects onto the ordered array of magnetic one-dimensional nanostructures. An alternating current source is electrically coupled to the magnet so that the magnet projects the magnetic field with a frequency onto the ordered array of magnetic one-dimensional nanostructures to differentiate the stem cell.

According to a further embodiment, there is a method for forming a system for differentiation of a stem cell. A scaffold, comprising an ordered array of magnetic one-dimensional nanostructures, is provided. A magnet is arranged proximate to the scaffold so that a magnetic field produced by the magnet projects onto the ordered array of magnetic one-dimensional nanostructures. An alternating current source is electrically coupled to the magnet so that the magnet projects the magnetic field with a frequency onto the ordered array of magnetic one-dimensional nanostructures to differentiate the stem cell.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1 is a flow diagram of a method for culturing a stem cell according to embodiments;

FIGS. 2A and 2B are schematic diagrams of a method for culturing a stem cell according to embodiments;

FIG. 3A is a schematic diagram of a system for culturing stem cells according to embodiments;

FIG. 3B is a schematic diagram of the behavior of a magnetic one-dimensional nanostructure to an applied magnetic field according to an embodiment;

FIG. 4 is a flow diagram of a method for forming a system for culturing stem cells according to an embodiment;

FIG. 5A is a scanning electron micrograph of mesenchymal stem cells cultured on a nanowire scaffold illustrating the mesenchymal stem cells adopting a contracted shape after two days of culturing; and

FIG. 5B is a scanning electron micrograph of mesenchymal stem cells cultured on a nanowire scaffold illustrating the focal adhesion points forming around the nanowires after two days of culturing.

### DETAILED DESCRIPTION

The following description of the exemplary embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to the terminology and structure of nanostructured magnetic scaffold.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

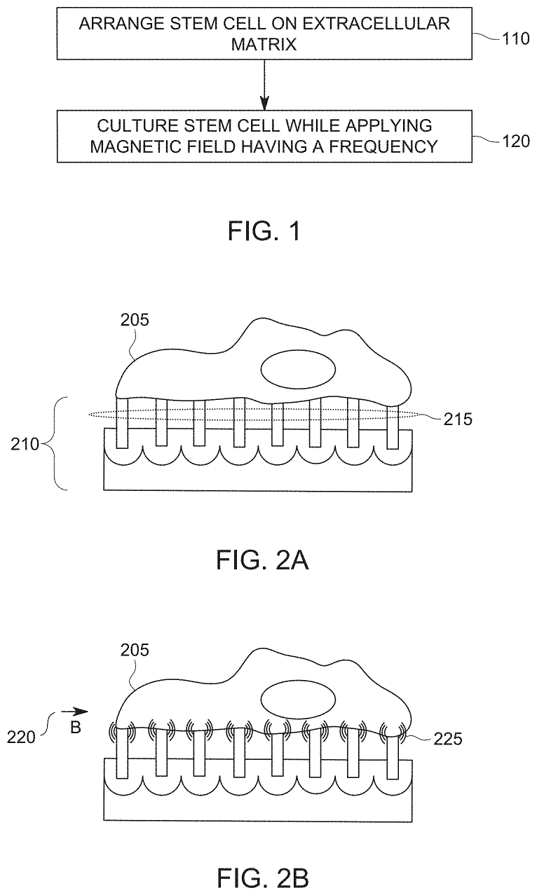

FIG. 1 is a flow diagram of a method for culturing a stem cell according to embodiments, which will be described in connection with the schematic diagrams of FIGS. 2A and 2B. As illustrated in FIG. 2A, a stem cell 205 is arranged on a scaffold 210 comprising an ordered array of magnetic one-dimensional nanostructures 215 (step 110). As illustrated in FIG. 2B, the stem cell 205 is then cultured while applying a magnetic field 220 having a frequency to the ordered array of magnetic one-dimensional nanostructures 215 to differentiate the stem cell 205 (step 120). As illustrated in FIG. 2B, the magnetic field 220 is applied in a direction perpendicular to the array of ordered array of one-dimensional nanostructures 215. Further, as illustrated by reference number 225, the one-dimensional nanostructures oscillate due to the magnetic field 220 in correspondence with the frequency of the magnetic field. The frequency of the magnetic field 220 should be a low frequency, such as, for example, 0.1-10 Hz.

During culturing, the applied magnetic field having a frequency does not have to be constantly applied to the scaffold 210. In one example, the magnetic field having a frequency is applied for twelve hours and is not applied for another twelve hours in a twenty-four hour period. This alternating application of the magnetic field having a frequency can be repeated for each twenty-four hour period during culturing.

As used herein, an ordered array of one-dimensional nanostructures means that there is a pattern of specific inter-nanostructure distance and nanostructure location across an area, such as an electrode or substrate. The one-dimensional nanostructures can be nanowires or nanorods, depending upon the aspect ratio of the one-dimensional nanostructures.

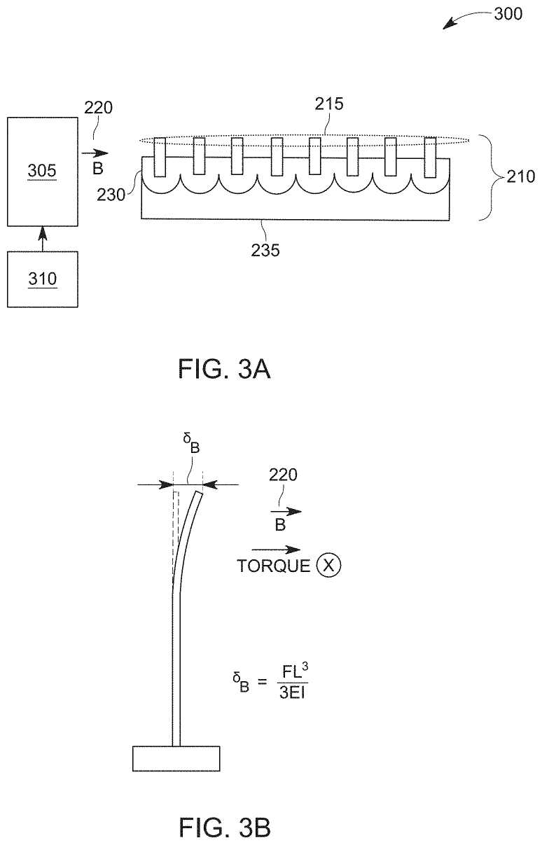

FIG. 3A is a schematic diagram of a system for culturing stem cells according to embodiments. The system 300 includes a scaffold 210 comprising an ordered array of magnetic one-dimensional nanostructures 215. A magnet 305 is proximate to the scaffold 210 so that a magnetic field 220 produced by the magnet 305 projects onto the ordered array of magnetic one-dimensional nanostructures 215. In one embodiment, the magnet 305 can be a projected field electromagnet, such as the GMW 5201 projected field electromagnet from GMW Associates. An alternating current source 310 is electrically coupled to the magnet 305 so that the magnet projects the magnetic field 220 with a frequency onto the ordered array of magnetic one-dimensional nanostructures 215 so that the stem cell 205 differentiates during culturing. Specifically, as illustrated in FIG. 3B, when a magnetic one-dimensional nanostructure is exposed to a magnetic field \vec{B} 220, a torque generated as the magnetic moment along the axis of the magnetic one-dimensional nanostructure aligns to the direction of the magnetic field \vec{B} 220. As also illustrated in FIG. 3A, the magnetic one-dimensional nanostructures are embedded in an aluminum oxide material 230, which is formed from an aluminum substrate 235. In an embodiment, the magnetic one-dimensional nanostructures can have a portion of, for example, 2-3 μm that are exposed above the aluminum oxide material 230.

Assuming, for example that the magnetic one-dimensional nanostructures have a length of 2-3 μm, a diameter averaging 33 nm, comprise iron, that the magnetic field has a force of 250 mT, and employing an end loaded cantilever beam model, the unloaded deflection δ_{B} at the free end of the magnetic one-dimensional nanostructure, due to the magnetic torque, was estimated to be approximately 100 nm. Specifically, elastic deflection defined as:

$${δ}_{B} = \frac{F {L}^{3}}{3 EI} , \qquad ( 1 )$$

where F is the force of the magnetic field that is applied to the system, L is the length of the beam or NW, E is the elastic modulus of the material (E=210 GPa for bulk Fe), and I is the moment of inertia, defined as:

$$I = \frac{π}{4} {r}^{4} , \qquad ( 2 )$$

where r=radius of the NW. The force F of the magnetic field that is applied to the free end of the magnetic one-dimensional nanostructure in equation (1) was modeled as the equivalent point load, which is the total force applied to the beam divided by its length:

$$Equivalent point load = \frac{F}{L} , \qquad ( 3 )$$

with the force F corresponding to the magnetic torque of a single magnetic one-dimensional nanostructure, the formula of which is:

τₘ=Mπr²lμ₀H, (4)

where μ₀H=B and M=M_{S}. Thus, solving for equation (1) yields an elastic deflection of approximately δ_{B}=100 nm.

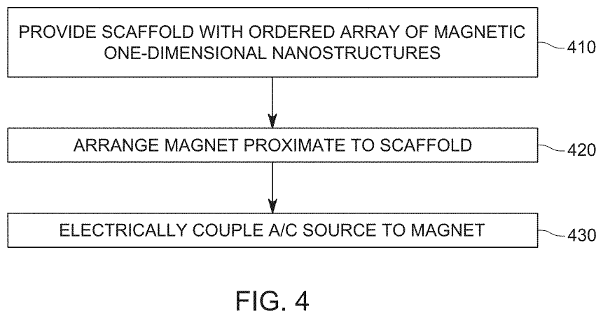

FIG. 4 is a flow diagram of a method for forming a system for culturing stem cells according to an embodiment. Initially, a scaffold 210 comprising an ordered array of magnetic one-dimensional nanostructures 215 is provided (step 410). A magnet 305 is arranged proximate to the scaffold 210 so that a magnetic field 220 produced by the magnet 305 projects onto the ordered array of magnetic one-dimensional nanostructures 215 (step 420). An alternating current source 310 is electrically coupled to the magnet 305 so that the magnet 305 projects the magnetic field 220 with a frequency onto the ordered array of magnetic one-dimensional nanostructures 215 to differentiate the stem cell 205 (step 430).

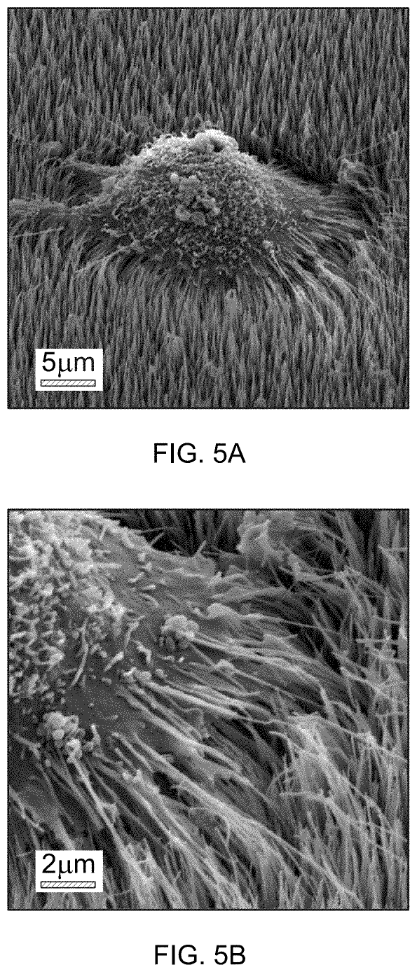

Culturing a stem cell in the manner described above results in the stem cell adopting a contracted shape with focal adhesion points forming around the magnetic one-dimensional nanostructures. For example, FIG. 5A illustrates a single, contracted mesenchymal stem cell after two days of culturing and FIG. 5B illustrates the formation of multiple focal adhesion points around the magnetic one-dimensional nanostructures as the mesenchymal stem cell attached to and grows on the scaffold, which causes the magnetic one-dimensional nanostructures to bend.

The inventors investigated whether the nanotopography of the magnetic one-dimensional nanostructures in the scaffold would lead to the expression of osteogenic markers. The extracellular matrix (ECM) protein osteopontin was selected due to its role in the early onset of osteogenesis. Immunofluorescence staining of osteopontin on mesenchymal stem cells cultured on the nanowires illustrated that no appreciable osteopontin fluorescent signal was observed after two days of culture, but a small increase was observed after one week of culture. However, the expression of osteopontin was significantly higher after two weeks of culture. Quantification of osteopontin fluorescence revealed a significant expression of this protein after both one week and two weeks. Immunofluorescence staining of the mesenchymal stem cell stem markers, CD105 and CD73 revealed the expression of both of these markers after two days of culture on the NWs, but the expression of CD73 was diminished after one week. The overall expression of both markers also appeared to be reduced compared to the negative control. These data suggest that mesenchymal stem cells retain these stem cell markers after two days of culture on the magnetic one-dimensional nanostructures, decreasing with time.

The expression of osteopontin observed here is most likely an effect of the stiffness and the nanotopography of the nanowires compared to routinely used tissue culture substrates. Thereby, the nanowire platform may more closely resemble the mechanical properties of the extracellular matrix and rearrange the cell cytoskeleton. The extracellular matrix influences the cell cytoskeleton via the signal transduction of integrins, which are cell membrane receptors that play an important role in osteogenesis, promoting the expression of markers such as Runx2, ALP and type 1 collagen. The decrease in the expression of CD105 and CD73 is in agreement with the onset of the expression of osteopontin, indicating a change in cell phenotype. In a similar setting, mesenchymal stem cells cultured on silicon nanowires also experienced an upregulation in the expression of osteogenic markers, type 1 collagen, Runx2, focal adhesion kinase, vinculin and several integrins. It was also proposed that the nanotopography of these nanowires activates Ca²⁺ channels and multiple mechanosensitive signaling pathways important in osteogenesis and chondrogenesis. Other nanotopographies, such as clusters of TiO₂ nanotubes and hydroxyapatite nanorods, have also been used as effective osteogenesis mediators of mesenchymal stem cells. There, the TiO₂ nanotubes increased the phosphorylation of the focal adhesion kinase, integrin clustering and the expression of osteopontin and osteocalcin, whereas the hydroxyapatite nanorods increased the expression of the osteogenic markers alkaline phosphatase (ALP), osteopontin and osteocalcin, as well as the formation of mineral nodules.

A low-frequency (0.1 Hz), 250 mT magnetic field was then applied to mesenchymal stem cells cultured on the nanowire scaffold, and whether there was osteopontin expression was determined after two days, one week and two weeks of culture under these field parameters. The magnetic field application significantly influenced the ability of the scaffold to induce the mesenchymal stem cells to differentiate down the osteogenic lineage when compared to the mesenchymal stem cells cultured on non-magnetically activated iron nanowires. Remarkably, osteopontin expression was observed as early as 2 days with the magnetic field. The stem cell marker CD105 was expressed only after two days of incubation and was quickly lost by the one-week time point, whereas the expression of CD73 was absent at all time points. Osteopontin is a cell-ECM interface structural protein, so it is expected to be ultimately located outside of the cell body at a certain time in the differentiation process. Indeed, staining for osteopontin was observed to be extracellular at both time points tested when mesenchymal stem cells were cultured on magnetically activated iron nanowires.

Overall, this suggests that the application of a magnetic field appears to enhance the osteogenic commitment of mesenchymal stem cells, as the expression of osteopontin was observed at a much earlier time point (two days) compared to control where no magnetic field was applied (two weeks). The loss of the stem cell markers CD105 and CD73 is consistent with the earlier osteogenic commitment observed with mesenchymal stem cells cultured on magnetically activated Fe NWs than those where no magnetic field was applied.

Document [1] demonstrated how focal adhesion points are formed on and around the magnetic nanowires, rearranging the cell cytoskeleton in the process. Given the critical role that the integrin-mediated focal adhesion kinase plays in the differentiation of mesenchymal stem cells in nanotopography-modulated mechanotransduction, the most likely scenario that explains an earlier onset of osteopontin expression on mesenchymal stem cells cultured on a magnetically activated nanowire scaffold is a positive effect on the formation of focal adhesion points. Thus, nanowire deflection could be promoting osteopontin expression through focal adhesion point modulation.

As we be appreciated from the discussion above, a biocompatible scaffold made of vertically arranged magnetic one-dimensional nanostructures that can be wirelessly activated to promote the differentiation of stem cells is provided. The nanotopography of the scaffold was sufficient to induce expression of the osteogenic marker, osteopontin, as early as one week of culture. When a low frequency magnetic field was applied in a direction perpendicular to the magnetic one-dimensional nanostructures in order to offer additional mechanical stimuli to the cells, the results revealed a remarkably faster onset of osteopontin expression, taking place after two days of culture. Culturing the mesenchymal stem cells on these biocompatible magnetic one-dimensional nanostructures, with or without magnetic activation, significantly brings down the time needed to induce osteogenic differentiation compared to the chemical methods traditionally used. The disclosed system shows potential for the targeting of mesenchymal stem cell differentiation in the context of tissue engineering and bone formation therapies and has potential for in-vivo applications in the future.

The disclosed embodiments are particularly advantageous because they can achieve differentiation of stem cells in as little as two days, whereas other techniques, such as that disclosed in Document [1] require a much longer time, such as two weeks. It is noted that the conclusion section of Document [1] mentions further exploration of the ability of iron nanowires to respond to a magnetic field but does not provide any details as to what this further exploration would involve. In contrast, the inventors have recognized that by applying a magnetic field having a frequency during culturing, as well as by applying the magnetic field perpendicular to the magnetic one-dimensional nanostructures while culturing, is particularly effective at reducing the time for stem cell differentiation. The use of a magnetic field having a frequency and applying it perpendicular to the magnetic one-dimensional nanostructures is new and non-obvious in view of Documents [1] and [2]. Document [2] relates to the application of a uniform field of 200 mT on fibroblast cells cultured on microposts embedded with magnetic nanowires. Fibroblast cells do not have the potential to differentiate like mesenchymal stem cells do, and thus Document [2] would not have provided any direction to one skilled in the art to use a magnetic field having a frequency to differentiate mesenchymal stem cells as disclosed herein. Further, the microposts in Document [2] are stiffer than the disclosed magnetic one-dimensional nanostructures, and thus the behavior of the microposts is not directly relatable to the behavior of the disclosed magnetic one-dimensional nanostructures. Additionally, the fabrication process of the microposts is significantly different than the fabrication of magnetic one-dimensional nanostructures, and in particular the template method discussed in Document [2] cannot be used to produce magnetic one-dimensional nanostructures.

Although some embodiments have been described in connection with differentiating mesenchymal stem cells into osteoblasts, the disclosed embodiments can be employed with other types of stem cells and differentiating them into other cell types.

The disclosed embodiments provide a nanostructured magnetic scaffold for controlling stem cell differentiation. It should be understood that this description is not intended to limit the invention. On the contrary, the exemplary embodiments are intended to cover alternatives, modifications and equivalents, which are included in the spirit and scope of the invention as defined by the appended claims. Further, in the detailed description of the exemplary embodiments, numerous specific details are set forth in order to provide a comprehensive understanding of the claimed invention. However, one skilled in the art would understand that various embodiments may be practiced without such specific details.

Although the features and elements of the present exemplary embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### CITED DOCUMENTS

[1] Mesenchymal Stem Cells Cultured on Magnetic Nanowire Substrates by J. E. Perez, T. Ravasi, J. Kosel, Nanotechnology 2016, 28, 55703.
[2] Magnetic Microposts as an Approach to Apply Forces to Living Cells by N. J. Sniadecki, A. Anguelouch, M. T. Yang, C. M. Lamb, Z. Liu, S. B. Kirschner, Y. Liu, D. H. Reich, C. S. Chen, Proc. Natl. Acad. Sci. U.S.A 2007, 104, 14553.

1. A method for differentiation of a stem cell, the method comprising:
arranging the stem cell on a scaffold comprising an ordered array of magnetic one-dimensional nanostructures; and
culturing the stem cell while applying a magnetic field having a frequency to the ordered array of magnetic one-dimensional nanostructures to differentiate the stem cell.

2. The method of claim 1, wherein the ordered array of magnetic one-dimensional nanostructures are arranged vertically with respect to a substrate of the scaffold and the magnetic field is applied horizontally to the ordered array of magnetic one-dimensional nanostructures.

3. The method of claim 1, wherein the stem cell is a mesenchymal stem cell that is differentiated into an osteogenic cell.

4. The method of claim 3, wherein osteopontin expression is observable after two days of culturing using the magnetic field.

5. The method of claim 1, wherein each day of the culturing involves a first period of time in which the magnetic field is applied and a second period of time in which the magnetic field is not applied.

6. The method of claim 5, wherein the first and second periods of time are both twelve hours.

7. The method of claim 1, wherein the frequency of the magnetic field is 0.1-10 Hz.

8. The method of claim 7, wherein the magnetic field has a force of 250 mT.

9. A system for differentiation of a stem cell, the system comprising:
a scaffold comprising an ordered array of magnetic one-dimensional nanostructures;
a magnet proximate to the scaffold so that a magnetic field produced by the magnet projects onto the ordered array of magnetic one-dimensional nanostructures; and
an alternating current source electrically coupled to the magnet so that the magnet projects the magnetic field with a frequency onto the ordered array of magnetic one-dimensional nanostructures to differentiate the stem cell.

10. The system of claim 9, wherein
the scaffold comprises a substrate from which each magnetic one-dimensional nanostructure of the ordered array of magnetic one-dimensional nanostructures are arranged vertically with respect to the substrate of the extracellular matrix, and
the magnet is proximate to the scaffold so that the magnetic field is applied horizontally to the ordered array of magnetic one-dimensional nanostructures.

11. The system of claim 9, wherein the magnetic one-dimensional nanostructures are biocompatible.

12. The system of claim 11, wherein the magnetic one-dimensional nanostructures comprise iron or an iron alloy.

13. The system of claim 9, wherein the alternating current source has an output frequency of 0.1-10 Hz.

14. The system of claim 13, wherein the magnetic field has a force of 250 mT.

15. The system of claim 9, wherein each one-dimensional nanostructure of the ordered array of one-dimensional nanostructures is partially in an alumina layer.

16. A method for forming a system for differentiation of a stem cell, the method comprising:
providing a scaffold comprising an ordered array of magnetic one-dimensional nanostructures;
arranging a magnet proximate to the scaffold so that a magnetic field produced by the magnet projects onto the ordered array of magnetic one-dimensional nanostructures; and
electrically coupling an alternating current source to the magnet so that the magnet projects the magnetic field with a frequency onto the ordered array of magnetic one-dimensional nanostructures to differentiate the stem cell.

17. The method of claim 16, wherein the magnet is arranged proximate to the scaffold so that the magnetic field is applied horizontally to the ordered array of magnetic one-dimensional nanostructures.

18. The method of claim 16, further comprising:
adjusting the alternating current source so that it outputs a current having a frequency of 0.1-10 Hz.

19. The method of claim 16, further comprising:
adjusting a voltage output by the alternating current source so that the magnet applies the magnetic field with a force of 250 mT.

20. The method of claim 16, wherein the magnetic one-dimensional nanostructures are biocompatible.
